(19) United States
(12) Patent Application Publication
(10) Pub. No.: US 2016/0121032 A1
(43) Pub. Date: May 5, 2016

(54) NEGATIVE PRESSURE WOUND THERAPY DRESSING AND DRAINAGE APPARATUS AND SYSTEM

(71) Applicant: Shakam LLC, Columbus, OH (US)

(72) Inventors: Jay Schwirian, Monongahelia, PA (US); Bradley Yakam, New Albany, OH (US)

(73) Assignee: Shakam LLC, Columbus, OH (US)

(21) Appl. No.: 14/926,388

(22) Filed: Oct. 29, 2015

Related U.S. Application Data
(60) Provisional application No. 62/072,130, filed on Oct. 29, 2014.

(51) Int. Cl. A61M 1/00; A61F 13/02

(52) U.S. Cl. CPC A61M 1/0088; A61F 13/0216; A61F 2013/0017

(57) ABSTRACT

A negative pressure wound therapy dressing and drainage apparatus includes: (1) a semi-permeable cover sheet adapted to cover a patient's wound; (2) a porous dressing adapted to be positioned between the semi-permeable cover sheet and the patient's wound; and (3) a drainage connection configured to be attached to the semi-permeable cover sheet including (a) a novel suction port in fluid communication with the porous dressing positioned within the interior of the semi-permeable cover sheet when attached to the semi-permeable cover sheet, and (b) tubing coupled to the suction port at one end and adapted to be connected to a suction source at an opposing end. The novel suction port includes a suction port inlet adapted to be secured to the semi-permeable cover sheet, a suction port outlet secured to the tubing and oriented generally perpendicular to the suction port inlet and a conduit extending between the suction port inlet and suction port outlet, where the inner surface of the conduit has smooth transition from the suction port inlet to the suction port outlet.

### RELATED U.S. APPLICATION DATA

This application claims the benefit of U.S. Provisional Application No. 62/072,130, filed on Oct. 29, 2014, the disclosure of which is incorporated herein by reference.

### BACKGROUND OF THE INVENTION

The current disclosure pertains to negative pressure wound therapy dressings and drainage systems. Negative pressure wound therapy (NPWT)—also called vacuum-assisted closure—may remove exudate, may help reduce bacterial growth, and may promote blood flow and granulation formation in open wounds. First, a foam dressing is placed in the wound and the wound is covered with an occlusive dressing. Then a suction port is placed on the covering and tubing is attached between the suction port and a pump, which creates sub-atmospheric pressure in the wound. Application of negative pressure to the dressing around the wound has been found to assist in healing the wound promoting blood flow to the area, stimulating the formation of granulation tissue, and encouraging the migration of healthy tissue over the wound. The suction port allows for wound exudates and other fluids to be drawn from the dressing to stimulate healing of the wound.

### SUMMARY

The current disclosure provides a negative pressure wound therapy dressing and drainage apparatus; and provides a new suction port design for such an apparatus. The apparatus comprises a semi-permeable cover sheet for covering a patient's wound, a porous dressing positioned between the semi-permeable cover sheet the patient's wound, and a fluid drainage connection attached to the semi-permeable cover sheet. The drainage connection includes a suction port in fluid communication with the porous dressing positioned within the interior of the semi-permeable cover sheet and a lumen coupled to the suction port at one end and adapted to be connected to a suction source (such as a pump) at an opposing end. The suction port is in the form of a conduit and having a suction port inlet secured to the semi-permeable cover sheet and a suction port outlet secured to the lumen and oriented generally perpendicular to the suction port inlet, where the inner surface of the conduit has a smooth transition from the suction port inlet to the suction port outlet.

In the more detailed embodiment the suction port inlet has an opening with an area larger than the area of the suction port outlet opening. Alternatively, or in addition, the upper inner surface of the conduit has a parabolic shape in axial cross-section, where the peak of the parabola is distal from the suction port outlet. In a more detailed embodiment the upper inner surface of the conduit has a step-free transition to an inner surface of the lumen. Alternatively or in addition, the suction port inlet opening widens with the distance from the suction port outlet to at least a certain point. In a more detailed embodiment, the suction port inlet narrows inward from the certain point. Alternatively or in addition, the suction port inlet opening is substantially triangular in shape with rounded corners.

In a further detailed embodiment, the suction port outlet may be molded to the lumen. In a more detailed embodiment, the suction port is a unitary component molded from thermoplastic polyurethane. In a further detailed embodiment the unitary suction port component is over-molded to the lumen.

### BRIEF DESCRIPTION OF THE DRAWINGS

FIG. 1 is a schematic diagram view of an exemplary negative pressure wound therapy dressing and drainage apparatus according to the current disclosure;

FIG. 2. is a perspective view of an exemplary suction port for a negative pressure wound therapy dressing and drainage apparatus according to the current disclosure;

FIG. 3 is an elevational cross-sectional view of the exemplary suction port of FIG. 2 taken along the axis of the outlet port;

FIG. 4 is an elevational side view of the exemplary suction port of FIGS. 2 and 3;

FIG. 5 is an elevational view of the outlet port end of the exemplary suction port of FIGS. 2-4;

FIG. 6 is a bottom view of the exemplary suction port of FIGS. 2-5;

FIG. 7 is a perspective view of an exemplary mold for molding the exemplary suction port of FIGS. 2-6;

FIG. 8 is a perspective interior view of the bottom portion of the exemplary mold of FIG. 7;

FIG. 9 is a perspective interior view of the top portion of the exemplary mold of FIG. 7;

FIG. 10 is a schematic representation of an adhesive design for the exemplary suction port of FIGS. 2-6;

FIG. 11 illustrates a fluid velocity profile for the exemplary suction port of FIGS. 2-6; and

FIG. 12 illustrates a fluid velocity profile for a prior art suction port.

### DETAILED DESCRIPTION

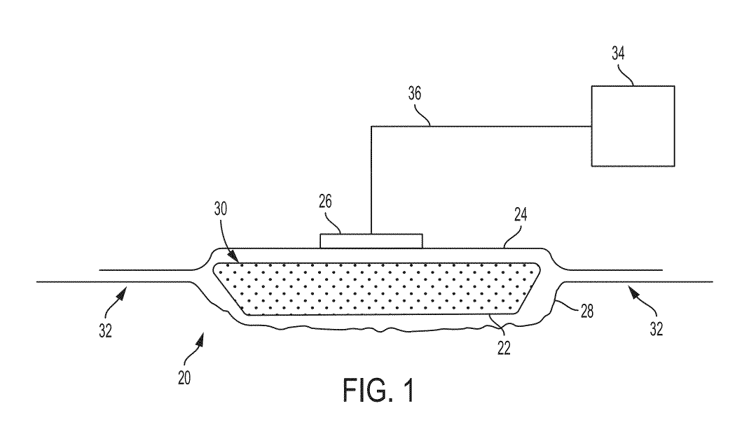

As shown in FIG. 1, a wound therapy dressing and drainage apparatus 20 comprises a porous substrate 22, which can be a foam material such as a polyurethane foam or can be some other porous material such as a gauze felt or other suitable material; a semi-permeable adhesive cover 24; and a suction port 26. The porous substrate 22 is positioned against the wound 28 and the semi-permeable cover 24 is placed over the porous substrate 22 and the patient's wound 28 such that the porous substrate 22 lies within a wound interior 30 provided between the semi-permeable cover 24 and the patient's wound 28. The cover 24 also extends beyond the wound 28 to healthy portions of the patient's skin 32 adhering thereto so as to form a sealed interior 30. Suction port 26 is in fluid communication with a suction source 34, such as a pump, via tubing 36. Operation of the suction source may provide a vacuum to the interior 30, thereby allowing the suction port 26 to draw fluids and other materials from the interior 30.

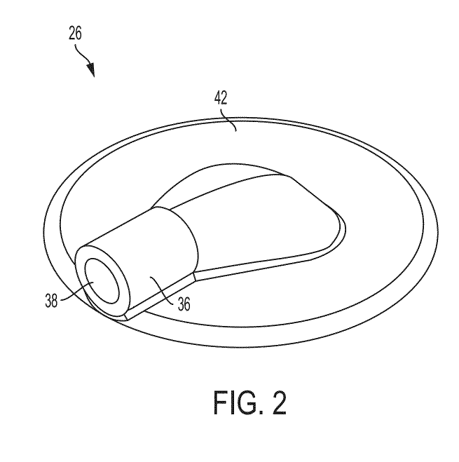

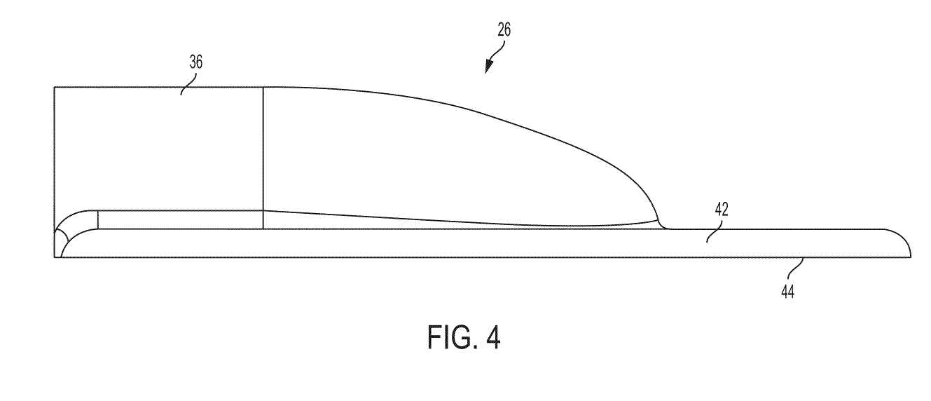

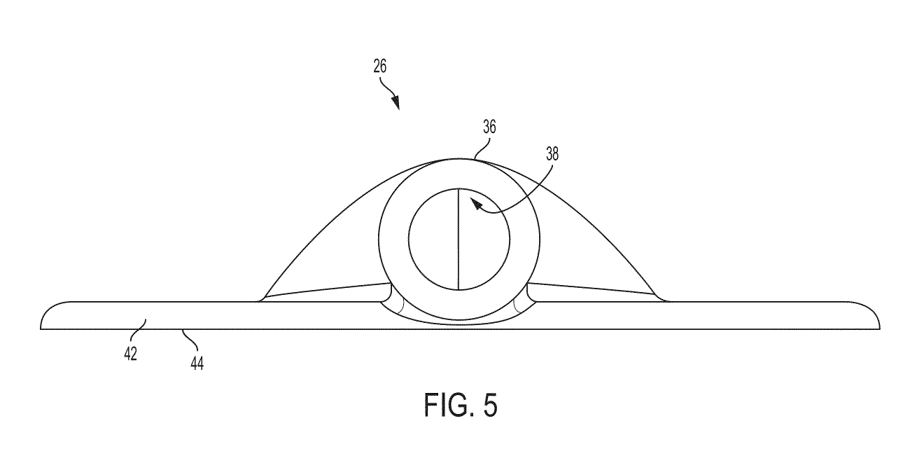

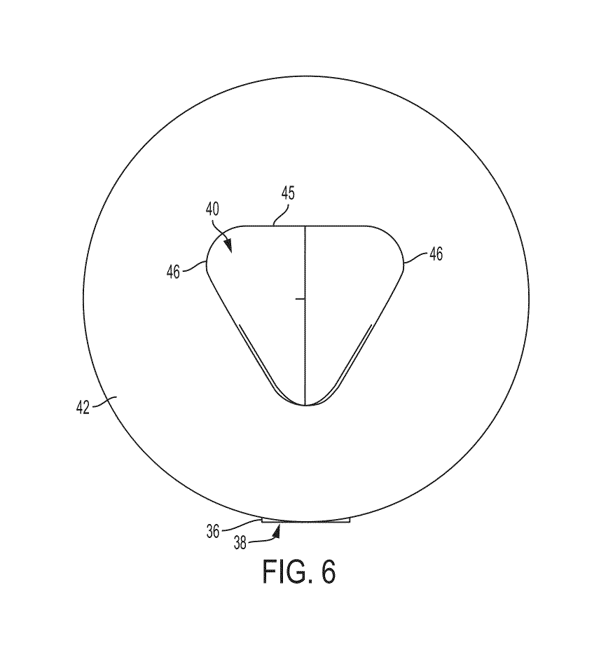

As shown in FIGS. 2 through 6, the suction port 26 includes a suction port outlet 38 and a suction port inlet 40. The axis of the fluid suction port outlet 38 is generally perpendicular to the axis of the suction port inlet 40. In an embodiment, as will be described below, the suction port outlet 38 comprises a leading end of tubing 36 co-molded onto the remainder of the suction port component 26. The suction port component 26 also includes a circular, planar flange 42 encircling the suction port inlet 40. The flange 42 has a planar bottom surface 44 that is coplanar with the opening of the suction port inlet 40. As shown specifically in FIGS. 2 and 6, the flange 44 extends radially out from the opening of the suction port inlet 40, so that the suction port inlet 40 is centralized with respect to the flange 42.

Referring specifically to FIG. 6, the suction port inlet 40 opening is generally in the shape of a triangle with rounded corners. The base 45 of the triangle defining the suction port inlet 40 opening is distal from the suction port outlet 38 so that the width of the suction port inlet opening generally increases from the distance from the suction port outlet 38 at least until a certain point 46 where the corners of the triangle round inwardly towards the base of the triangle 45. The area of the suction port inlet 40 is substantially larger than the area of the suction port outlet 38, which is in the form of a circle defined by the interior diameter of the lumen 36.

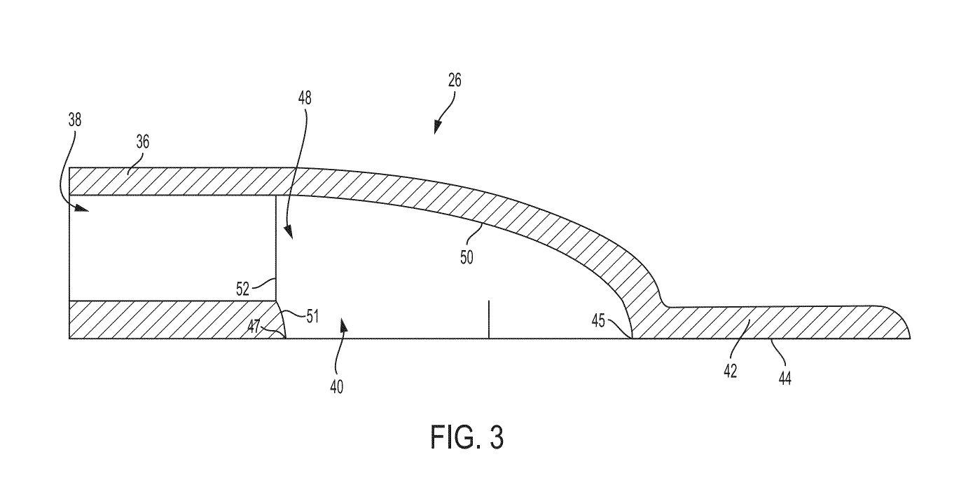

Referring now specifically to FIG. 3, the suction port 26 defines a conduit 48 extending between the suction port inlet 40 and the suction port outlet 38. The conduit 48 includes an upper inner surface 50 that extends from the base 45 of the triangular suction port inlet opening and curves in a parabolic shape (where the peak of the parabola is approximate the base 45 point) from the triangle base 45 up and back to an upper surface of the outlet 38 in an integration area 52 between the suction port body and the tubing 36. As shown by the upper inner surface 50, such a curve provides a step-free transition from the upper inner surface of the suction port 26 to the lumen 36. The lower inner surface 51 is a tighter curve extending from the triangle peak 47 up and back to a lower surface of the outlet 38 in integration area 52, and also provides a step-free transition.

Such a design for the suction port 26 provides a relatively large inlet opening (as compared to the outlet opening) over the porous material 22 that would be placed beneath it. This larger volume of the conduit 48 at the suction port inlet 40 opening decreases the ability for fibrins, proteins, and/or sediments that can accumulate in the porous material 22 and clog or seal off the suction port. With such a large suction port opening, it would take a larger mass of such materials to clog the suction port of the current disclosure. Further, the gradual slope of the conduit 48 allows for greater velocity of fluids passing there through, less turbulence or swirling of the fluids and a lower profile. By moving the fluids at a greater velocity and with less turbulence, sediments in the fluid have a lesser chance to congeal at the opening or in the tubing. Further, a lower profile will reduce the likelihood of snagging on the bed or linens when a patient moves, turns or is transferred out of the bed.

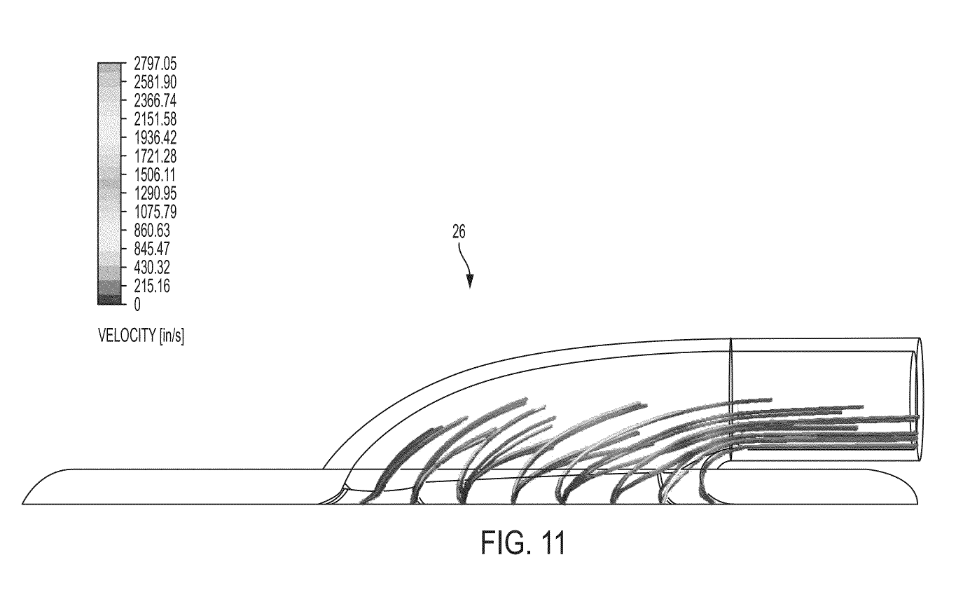

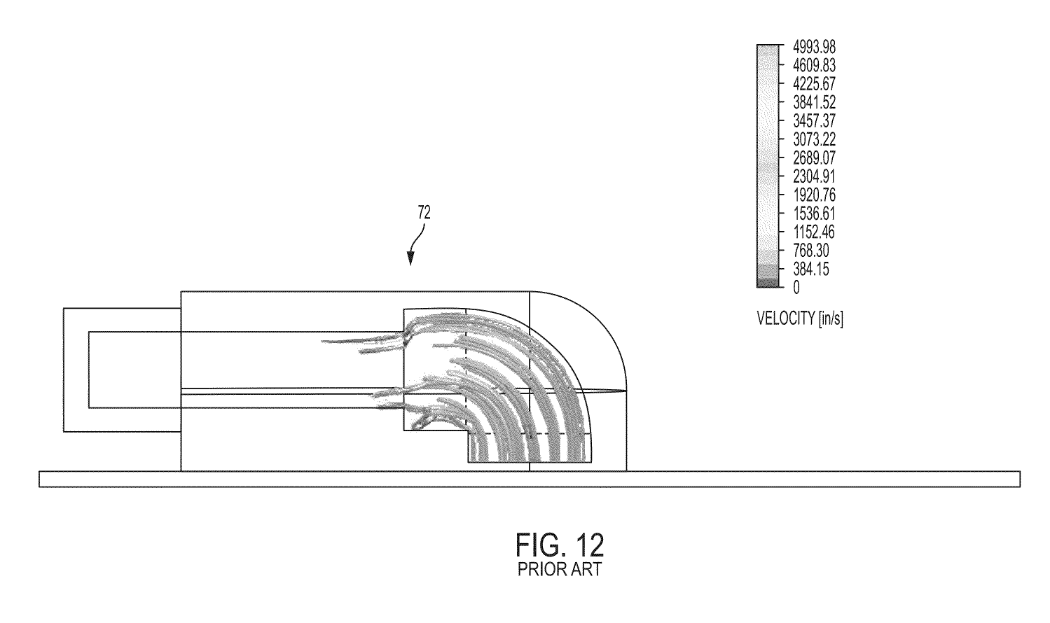

FIG. 11 shows a fluid velocity profile of fluids as they are sucked from the porous material 22 and into the tubing 36 using the exemplary suction port 26 as described herein. In comparison, FIG. 12 shows a fluid velocity profile of a prior art suction port. As can be seen in the comparison, the fluids passing through the exemplary suction port 26 have a greater velocity and experience less turbulence or swirling as compared to the velocity profile of fluids passing through the prior art suction port 72.

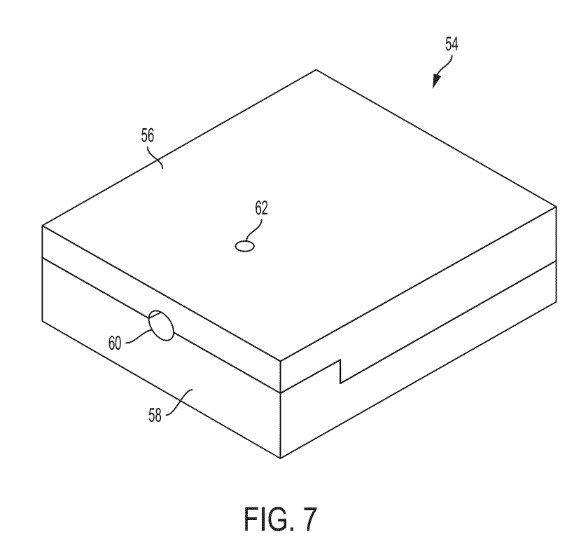

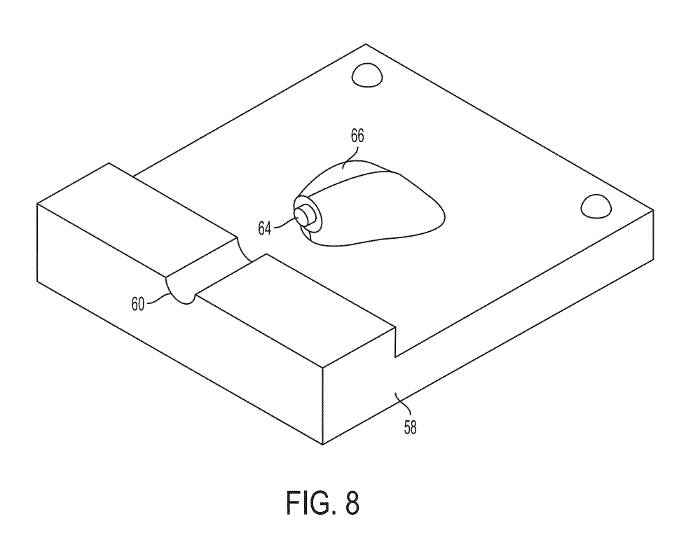

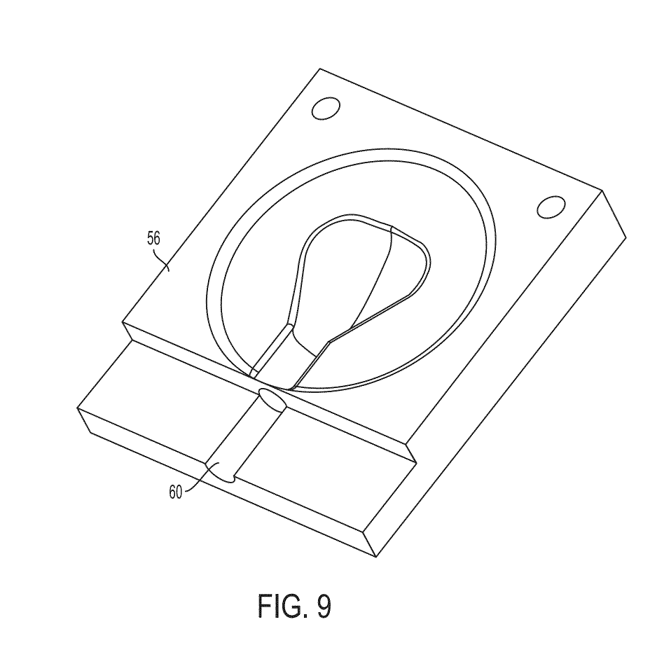

In an embodiment, rather than molding the suction port inlet 26 as a separate piece and then gluing the tubing 36 onto the suction port 26, the suction port 26 and tubing 36 may be over-molded together. Referring to FIGS. 7 through 9, a mold 54 for such a molding process is illustrated. The mold 54 includes a top mold portion 56 and a bottom mold portion 58. The mold 54 includes a cylindrical guide 60 for receiving the tubing therein, and also includes an opening 62 for receiving the molten molding material. As shown in FIG. 8, the guide 60 seats the tubing thereon. Before injecting the molten material into the opening 62, the tubing (not shown in this figure) is inserted into the guide 60 and inserted onto a boss 64 extending from a positive representation 66 of the inlet port portion of the conduit 48 (the boss is shaped as a cylinder sized to fit in the channel of the tubing). Once the tubing is placed in the mold 54, the hot molten material thermoplastic polyurethane material (such as Texin®) is injected into the mold at around 370° F. In an embodiment, the tubing and the molten material are the same thermoplastic polyurethane material, causing the molten material to melt the surface of the tubing and to become integrated as one piece when it cools. The result is an integral bond with no air leaks which provides an advantage over the prior art suction ports that struggle with air leaks by using the gluing methods.

In an embodiment, the Texin material cools into a transparent component. This allows the porous material 22 to be seen by a practitioner through the transparent suction port 26.

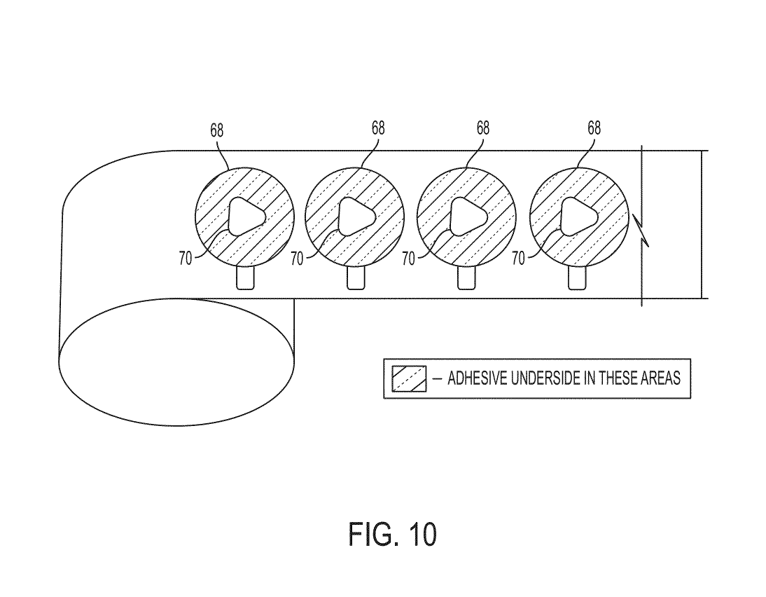

FIG. 10 illustrates a roll of adhesive stickers 68 adapted to be applied to the under surface 44 of the flange 42. These double-sided adhesive stickers 68 provide the ability for the suction port to adhere to the semi-permeable cover 24. As shown in FIG. 10, each adhesive sticker 68 includes a triangular opening 70 corresponding to the opening of the suction port inlet 40.

While example embodiments have been set forth above for the purpose of disclosure, modifications of the disclosed embodiments as well as other embodiments thereof may occur to those skilled in the art. Accordingly, it is to be understood that the disclosure is not limited to the above precise embodiments and that changes may be made without departing from the express scope of the following claims. Likewise, it is to be understood that it is not necessary to meet any or all of the stated advantages or objects disclosed herein to fall within the scope of the disclosure, since inherent or unforeseen advantages may exist even though they may not have been explicitly discussed herein.

What is claimed is:

1. A negative pressure wound therapy dressing and drainage apparatus comprising:
a semi-permeable cover sheet adapted to cover a patient's wound;
a porous dressing adapted to be positioned between the semi-permeable cover sheet and the patient's wound; and
a drainage connection configured to be attached to the semi-permeable cover sheet including (a) a suction port in fluid communication with the porous dressing positioned within the interior of the semi-permeable cover sheet when attached to the semi-permeable cover sheet, and (b) tubing coupled to the suction port at one end and adapted to be connected to a suction source at an opposing end;
the suction port having a suction port inlet adapted to be secured to the semi-permeable cover sheet, a suction port outlet secured to the tubing and oriented generally perpendicular to the suction port inlet and a conduit extending between the suction port inlet and suction port outlet, the inner surface of the conduit having smooth transition from the suction port inlet to the suction port outlet.

2. The negative pressure wound therapy dressing and drainage apparatus of claim 1, wherein the suction port inlet opening has an area larger than an area of the suction port outlet opening.

3. The negative pressure wound therapy dressing and drainage apparatus of claim 1, wherein an upper inner surface of the conduit has a parabolic shape in axial cross-section, the peak of the parabola being distal from the suction port outlet.

4. The negative pressure wound therapy dressing and drainage apparatus of claim 3, wherein the upper inner surface of the conduit has a step free transition to an inner surface of the tubing.

5. The negative pressure wound therapy dressing and drainage apparatus of claim 3, wherein the suction port inlet opening widens with a distance from the suction port outlet to at least a certain point.

6. The negative pressure wound therapy dressing and drainage apparatus of claim 5, wherein the suction port inlet opening narrows inward from the certain point.

7. The negative pressure wound therapy dressing and drainage apparatus of claim 5, wherein the suction port inlet opening is substantially triangular in shape with rounded corners.

8. The negative pressure wound therapy dressing and drainage apparatus of claim 3, wherein the suction port includes an attachment flange extending radially out from the suction port inlet.

9. The negative pressure wound therapy dressing and drainage apparatus of claim 8, wherein the flange is substantially planar and circular.

10. The negative pressure wound therapy dressing and drainage apparatus of claim 1, wherein the suction port outlet is molded to the tubing.

11. The negative pressure wound therapy dressing and drainage apparatus of claim 10, wherein the suction port is a unitary component molded from thermoplastic polyurethane.

12. The negative pressure wound therapy dressing and drainage apparatus of claim 11, wherein the unitary suction port component is overmolded to the tubing.

13. A suction port for a negative pressure wound therapy dressing and drainage apparatus comprising:
a suction port inlet adapted to be secured to a semi-permeable cover sheet of the negative pressure wound therapy dressing and drainage apparatus;
a suction port outlet adapted to be secured to a suction tubing of the negative pressure wound therapy dressing and drainage apparatus and oriented generally perpendicular to the suction port inlet; and
a conduit extending between the suction port inlet and suction port outlet, the inner surface of the conduit having smooth transition from the suction port inlet to the suction port outlet.

14. The suction port of claim 13, wherein the suction port inlet opening has an area larger than an area of the suction port outlet opening.

15. The suction port of claim 13, wherein an upper inner surface of the conduit has a parabolic shape in axial cross-section, the peak of the parabola being distal from the suction port outlet.

16. The suction port of claim 15, wherein the upper inner surface of the conduit has a step free transition to an inner surface of the tubing.

17. The suction port of claim 15, wherein the suction port inlet opening widens with a distance from the suction port outlet to at least a certain point.

18. The suction port of claim 17, wherein the suction port inlet opening narrows inward from the certain point.

19. The suction port of claim 17, wherein the suction port inlet opening is substantially triangular in shape with rounded corners.

20. The suction port of claim 15, wherein the suction port includes an attachment flange extending radially out from the suction port inlet.

21. The suction port of claim 12, wherein the flange is substantially planar and circular.

22. The suction port of claim 13, wherein the suction port outlet is molded to the tubing.

23. The suction port of claim 13, wherein the suction port is a unitary component molded from thermoplastic polyurethane.

24. The suction port of claim 23, wherein the thermoplastic polyurethane material is substantially transparent.

25. The negative pressure wound therapy dressing and drainage apparatus of claim 23, wherein the unitary suction port component is overmolded to the tubing.
